(19) United States
(12) Patent Application Publication
(10) Pub. No.: US 2004/0249326 A1
(43) Pub. Date: Dec. 9, 2004

(54) Water-hardening fixing material and method for using the same

(76) Inventors: Yoshikazu Matsumoto, Funabashi-shi Chiba-ken (JP); Shozo Ohnishi, Tokyo (JP)

Correspondence Address: Adams & Wilks, 31st Floor, 50 Broadway, New York, NY, 10004, US

(21) Appl. No.: 10/489,557

(22) PCT Filed: Sep. 27, 2002
(86) PCT No.: PCT/JP02/10019

(30) Foreign Application Priority Data
Sep. 28, 2001 (JP) 2001-299812

(51) Int. Cl. A61F 5/00

(52) U.S. Cl. 602/8; 602/6

(57) ABSTRACT

It is an object to obtain a water-curable supporting bandage which can be applied to even an affected part of complicated shape with sufficient time, when it is applied by winding it around an affected part such as bone fracture for fixing. A supporting bandage comprising a water-curable polyurethane pre-polymer composition held on the fabric material is applied to an affected part without water supply. Sufficient time can be taken for application of the supporting bandage to the affected part. After completion of the application, the polyurethane prepolymer is rapidly cured by supplying water from outside of the supporting bandage to obtain a sufficient strength.

The fabric material of the supporting bandage has from 15 to 33 apertures per cm² and a numerical opening rate of aperture of from 14 to 35%, and is flexible. This fabric material is covered with a water-curable polyurethane prepolymer composition comprising a polyurethane prepolymer containing a polyol and polyisocyanate, and a catalyst. In this polyurethane prepolymer composition, an ethylene oxide component of the polyol is contained in an amount of 12 wt % or higher.

### BACKGROUND OF THE INVENTION

1. Technical Field

The present invention relates to a water-curable supporting bandage useful for treatment in surgery and orthopedic surgery fields by fixing an injured or diseased part of a patient, i.e., an affected part, and a method for using it.

2. Background Information

Water-curable supporting bandage obtained by coating a water-curable polyurethane prepolymer composition on a fabric material (substrate) of a tape-like or sheet-like form and sealed in a moisture-impermeable bandage,are commercially available. To use such bandages, the water-curable supporting bandage is immersed or dipped in water and applied it to the affected part, it undergoes curing or hardening in a short time, whereby the affected part can be fixed and sufficient strength can be obtained. Further, when used, no special equipment is required and no contamination or odor is generated. Furthermore, from the viewpoint of patients, fixing in a short time is possible, air permeability is good and X-ray photograhing can be made, whereby it has been widely used in place of conventional plaster bandages.

When the water-curable supporting bandage is-immersed in water to bring contact with water, the polyurethane prepolymer composition starts a curing reaction. When this water-curable supporting bandage is applied to the affected part, the flexible water-curable supporting bandage gradually hardens. When the curing reaction further proceeds, it possesses a strength such that it undergoes no deformation even if a load is applied to some extent, and when the reaction is completed, it forms a rigid member having a high strength. Since the water-curable supporting bandage for orthopedic surgery is required to cure early after the application to the affected part, the water-curable polyurethane prepolymer composition is required to have a high activity to water.

The process for applying the water-curable supporting bandage to the affected part of a patient can include the following steps:

(1) the step of applying the water-curable supporting bandage which is already contacted with water to the affected part (the time period available for this will hereinafter be referred to as the “working time”).

(2) the step of modifying the water-curable supporting bandage partially so as to make it suited to the affected part, i.e., conduct a so-called modeling (this time period will hereinafter be referred to as the “modeling time”),

(3) the step wherein after the completion of the modeling,the curing of the water-curable supporting bandage proceeds until deformation is no longer seen even if a load is applied thereto (this time period will be hereinafter referred to the “weighi bearing time”; and

(4) the step in which the curing of the water-curable supporting bandage further proceeds into a completely cured state.

Having considered the relationship between the strength of the water-curable supporting bandage and the time in the above steps, the following becomes clear. The step (1) is an operation of applying the water-curable supporting bandage to the affected part, and in a case where it is applied to a site having a particularly complicated shape, it is necessary to take a certain length of time In this step, if the curing speed of the water-curable polyurethane prepolymer composition is too fast, it is impossible to apply it appropriately to the required position. Accordingly, in the time period necessary for the application,it is desirable that the strength of the water-curable supporting bandage is kept as low as possible.

Next, with regard to the modeling step (2), the water-curable supporting bandage applied to the substantially adequate position in the step (1) is partially modified to fit it into the affected part. If the strength of the water-curable supporting bandage is kept at a low level, when a force is applied to the water-curable suppoprting bandage for modification so as to conduct modeling into an accurate shape, and then the force is removed, it returns to the shape before the modification due to the restoring force or elasticity of the water-curable supporting bandage. When this occurs, too much time and labor can be required for finishing the final modeling of the bandage. Accordingly, it is desirable that the strength of the water-curable supporting bandage gradually increases in the modeling step.

In the step (3), the water-curable supporting bandage after completion of the modeling is disposed in an appropriate position throughout the affected part. It is therefore required to hold the affected part so that the affected part does not move until the water-curable supporting bandage further cures and undergoes no deformation even if ordinary load is applied. Accordingly,it is desirable that the strengthof the water-curable supporting bandage increases as fast as possible (hereinafter the beginning of the curing until this step is referred to as “initial curing property”).

Further, in the step (4), in order to shorten the time during which both the patient and the doctor must be engaged or involved in the medical treatment, it is desirable for the water-curable supporting bandage to cure sufficiently and the strength of the cured bandage reaches the highest possible value.

Namely, throughout the steps (1) to (4), it is desirable that the water-curable supporting bandage has a curing reaction property such that the strength is kept at a low level during a handling period in which it is applied to the affected part, increases promptly when it enters the modeling step, and thereafter increases while continuously undergoing the curing, and finally reaches a high level.

Various water-curable supporting bandage which have been proposed in the conventional art are,as mentioned above, ones which are immersed in water at the time of use to let them hold water sufficiently,and then are wound around the affected part for application. As mentioned above,it is required to speed up the curability of the polyurethane prepolymer composition after the modeling step. If doing so, however, the curing steadily proceeds even during the application to the affected part, whereby the working time is shortened and it is difficult to apply it with sufficient time to complicated affected part, such being disadvantageous.

### SUMMARY OF THE INVENTION

It is an object of the present invention to provide a water-curable supporting bandage which can be applied to even the affected part of complicated shape with sufficient working time, and accelerates the curing property when it enters the modeling step, and is easily used and finally presents a sufficient strength.

In the present invention, a water-curable supporting bandage comprising a fabric material and a water-curable polyurethane prepolymer composition held on the fabric material, is as it is applied to the affected part. After completion of the application,the prepolymer is rapidly cured by supplying water from the outside of the bandage to obtain a sufficient strength. At this time, the fabric substrate of the bandage has from 15 to 33 apertures per cm² and a numerical opening rate of aperture of from 14 to 35%, and is flexible. This fabric substrate is covered with a water-curable polyurethane prepolymer composition comprising a polyurethane prepolymer containing a polyol and polyisocyanate, and a catalyst. In this polyurethane resin composition, an ethylene oxide component of the polyol is contained in an amount of 12 wt % or higher.

The water to be supplied after the water-curable supporting bandage is applied to the affected part, may be water solely, but if water containing a surface active agent is used, that can be evenly applied throughout the bandage, and the curing can be accelerated. Further,it is advantageous to supply the water by spraying it over the bandage applied to the affected part.

In order to accelerate the curability after the start of the modeling step, it is desirable that the polyurethane prepolymer composition appropriately emit (generates) heat. For this purpose, a compound which is capable of generating heat when it contacts water may be added to the polyurethane prepolymer composition. In such a case, when the modeling step operation is carried out by supplying water to the bandage applied to the affected part, the contact of this heat-generating compound with water can be further easily caused at the outermost layer of the rolled bandage. And, since the heat is supplied from the outermost layer toward the inner layer of the rolled bandage, the curing can further be accelerated.

### BRIEF DESCRIPTION OF THE DRAWINGS

FIG. 1 is a table showing materials used for the water-curable polyurethane prepolymer composition in examples and comparative examples of the present invention.

FIG. 2 is a table showing the formulation of the water-curable polyurethane prepolymer composition in examples of the present invention.

FIG. 3 is a table showing the formulation of the water-curable polyurethane prepolymer composition in comparative examples of the present invention.

FIG. 4 is a table showing the results of the respective measurements in examples of the present invention.

FIG. 5 is a table showing the results of the respective measurements in comparative examples of the present invention.

FIG. 6 is a graph showing the relation between the initial curability and the elapsed time in examples and comparative examples of the present invention.

### DETAILED DESCRIPTION OF THE PREFERED EMBODIMENTS

As the fabric material for the water-curable supporting bandge, it is possible to use knit, woven fabric, nonwoven fabric and the like obtained from materials which are flexible, have a low moisture content and a high tensile strength, and are inactive to the polyurethane prepolymer composition and easily wettable. For example, knit,woven fabric and nonwoven fabric using glass fibers, aramid fibers, polyester fibers, polyolefin fibers, polyamide fibers, polyacryl fibers, rayon fibers,cotton fibers and the like, may be mentioned. Raschel fabric using yarn of glass fibers or polyester fibers is particularly preferred, and the thickness is referably about from 0.08 to 5 mm.

This fabric material preferably has about from 15 to 33 apertures per cm² and a numerical opening rate of aperture of about from 14 to 35%. With apertures having the number of apertures of 34 or more per cm² and the numerical opening rate of aperture of less than 14%, the permiability of water applied by e.g., spraying is low. With apertures having the number of apertures of 14 or less per cm² and the numerical opening rate of aperture exceeding 35%, the water applied by e.g., spraying is not held in bandage and flows out. Accordingly, in both case, it is difficult to cure polyurethane prepolymer composition sufficiently. The numerical opening rate of aperture is the ratio of the area occupied by the spacing in a defined or unit area, and can be determined mechanically by taking an enlarged photograph of the fabric material and making image recognition of the photograph.

The polyurethane prepolymer for use in the present invention is the one which is obtained by reacting a polyol with a polyisocyanate and has an isocyanate group at its terminal end.

As the polyol, polyethylene glycol (PEG), and a random or block copolymer of ethylene oxide and propylene oxide are used. The average molecular weight of the polyol is desirably about 200 to 4,000. In case of a water-curable supporting bandage using polyol with the molecular weight less than 200, the rigidity thereof after it has cured is large,and bandage is leaded to hard and brittle properties. If the molecular weight is higher than 4,000, the rigidity is small, the strength of the bandage is sometimes insufficient. Further, together with above-mentioned polyols, well-known polyols can be used. For example, polypropylene glycol (PPG), bisphenol type diol (BP, BPE) and the like may be mixed appropriately for use. It is necessary that the ethylene oxide component of the polyol is contained in an amount of about 12 wt % or higher, preferably about 18 wt % or higher, in the polypolyurethane prepolymer composition.

As the polyisocyanate, well-known polyisocyanate may be used. For example, 4,4′-diphenylmethane diisocyanate, 2,4′-diphenylmethane diisocyanate, polymethylenepolyphenyl polyisocyanate, or the like,and not only these p-phenylene diisocyanate but also carbodiimide-modified polyisocyanate of them may be mentioned. These may be used singly or in combination of two or more of them. It is preferred to use 4,4′-di-phenylmethane diisocyanate, 2,4′-diphenylmethane diisocyanate and carbodiimide-modified polyisocyanate of them.

As the proportional ratio of a polyol and polyisocyanate to obtain the polyurethane prepolymer having an isocyanate group at its terminal end, the polyisocyanate is usually from 2 to 5 equivalents, preferably from 2.5 to 5 equivalents, per 1 equivalent of the polyol. The reaction of both is made by heating and stirring usually at about from 30 to 100° C., preferably at about from 50 to 80° C. The viscosity of the polyurethane prepolymer is usually about from 10 to 50 Pa·s, preferably about from 15 to 40 Pa·s at room temperature of 23° C.

As the catalyst, any of those catalysts having excellent storage stability should preferably be selected for use. Such catalyst which have been well-known includes catralyst such as dimorpholino diethylether, bis(2,6-dimethylmorpholino)diethylether, and substituted morpholino diethylether may be mentioned. These may be used alone or in combination of two or more of them.

After the application of the water-curable supporting bandage to the affected part, when curing is conducted by spraying water, the amount of the catalysts is preferably such that the after-mentioned gelling time of the polyurethane prepolymer composition is 70 seconds or less. For this purpose, the catalyst is preferably contained in an amount of about from 1.3 to 6 wt % in the composition. If the catalyst amount is less than 1.3 wt %,in the above-mentioned initial curability, the curability after spraying water can not sufficiently be improved. And within a range of the catalyst amount exceeding 4 wt % until 6 wt %, the strength is equal to as compared with the initial curability when the catalyst amount is 4 wt %, and if it exceeds 6 wt %, the strength of the bandage becomes to lower.

In the polyurethane prepolymer composition, a additive compound which is capable of generating heat when it contacts water (dissolution) may be used. As the compound which is capable of generating heat when it contacts water, various types may be used. For example, chlorides of metals such as calcium chloride and magnesium chloride, oxides of metals such as calcium oxide and zinc oxide, sulfates of metals such as calcium sulfate and magnesium sulfate, silica and the like may be mentioned.

In the polyurethane prepolymer composition, a stabilizer may appropriately be used. As the stabilizer, well-known benzoyl chloride, methanesulfonic acid, p-toluenesulfonic acid may be used. These stabilizers may likewise be used alone or in combination of two or more of them. The amount of the stabilizer is determined depending upon the catalyst amount, and usually about from 0.005 to 1 wt %, preferably about from 0.01 to 0.5 wt % of the polyurethane prepolymer composition. When it is less than 0.005 wt %, stabilization effect may not be obtained,and when it exceeds 1 wt %, the activity of the catalyst may be impaired.

In the polyurethane prepolymer composition of the present invention, as the case requires, a defoaming agent, an antioxidant, a viscosity modifier, an adhesion retardant, an ultraviolet absorber, colorants such as a pigment and a dye, fillers such as calcium carbonate, titanium dioxide, carbon black, clay and the like, may further be contained.

As the amount of the water to be supplied to the water-curable supporting bandage by spraying or the like, the curing can be made by-supplying a small amount at a level of about from 20 to 60 g/m², preferably at a level of about from 40 to 50 g/m². Further, it is preferred to add-a surface active agent to the water to be supplied. As the surface active ageant, any one of nonionic, anionic, cationic and amphoteric surface active agent may be used,but ones having a low skin stimulus property is desirable. For example, as the nonionic type, polyoxethylene alkylether type,and as the anionic type, sodium polyoxyethylene laurylether sulfate, poly-oxyethylene laurylether sulfate triethanolamine and the like are preferred. As the specific spraying method, the above predetermined amount of water is sprayed by a sprayer or an aerosol container filled with water additionally containing the surface active agent or containing no surface active agent.

For preparation of the polyurethane prepolymer composition, the catalyst, stabilizer and various additives may be added to the polyurethane prepolymer obtained from a polyol and polyisocyanate. Further, at the time of preparing the polyurethane prepolymer, together with the polyol and polyisocyanate, a part or whole of a catalyst, a stabilizer, calcium chloride and various additives may preliminarily be added. Further, after polyurethane prepolymer is coated on a fabric material, calcium chloride may be sprinkled and adhered on it.

The method for producing the water-curable supporting bandage by covering the fabric material with the polyurethane prepolymer composition,may be conducted well-known methods. For example,a method wherein the polyurethane prepolymer composition is coated on the fabric material by a roll in a room controlled at a low humidity. The obtained water-curable supporting bandage is desirably kept in a sealed container which is capable of blocking moisture.

When the water-curable supporting bandage is used, the sealed container is opened and the bandage is taken out, and this bandage is as it is applied by winding it around the affected part of patients. At this time, since water is not yet applied to the bandage, full-scale curing is not yet started although a slight curing might have been started by the moisture in air. Accordingly, it is possible to securely apply it with sufficient time even to an affected part of complicated shape.

After it is applied as mentioned above, by applying water by spraying water on the surface of the bandage, polyurethane prepolymer starts the reaction and begins curing. When a heat-generating material such as calcium chloride is contained in the bandage,the reaction is accelerated by further heat generation. When modeling is conducted in the progress of curing to make modification so as to fit it in the affected part, and the curing is further advanced, the affected part can be securely fixed.

The process of the curing of the water-curable supporing bandage is considered as follows. Namely, polyethylene glycol as the polyol content in the polyurethane prepolymer has a strong hydrophilicity. After the bandage is applied to the affected part of patient, when water is sprayed from the outside thereof, the water rapidly impregnates into the polyurethane prepolymer composition and starts the reaction with the isocyanate functional group. At this time, if a surface acive ageant is contained in the water to be sprayed, the permeability into the water-curable supporting bandage is increased and the water can be applied more evenly throughout it. Further, the permeability of water is also related to the above-mentioned number of apertures and numerical opening rate of aperture. Within a range where the number of apertures is from 14 to 34 per cm² of the fabric material and the numerical opening rate of aperture is from 13 to 35%, excellent results can be obtained. Preferably, the number of apertures from 15 to 33 and the numerical opening rate of aperture is from 14 to 33%, more pre-ferably, the number of apertures is from 20 to 25 per cm² and the numerical opening rate of apeture is from 18 to 27%.

If the sprayed water impregnates and is held in the bandage effectively,the catalyst in the polyurethane prepolymer composition accelerates the reaction of bewteen water and isocyanate group of polyurethane prepolymer composition, thereby advancing the curing. By the reaction heat by the reaction of water with isocyanate, the temperature is raised and the reaction rate is further increased, whereby the curing can be made from the outer layer toward the inner layer at the affected part side of wound bandage in a short time. And, since the ratio that water reaches the most affected part side (most inner side) of the wound bandage is low, the heat generation at the affected part side is small.

When the curing is made by a spraying method, with a polyurethane prepolymer using polypropylene glycol, the hydrophilicity is low and the permeability of water is small, whereby it is difficult to obtain the desired strength in a short time at a practical level. Accordingly,in order to obtain a preferred water permeability, the ethylene oxide component in the polyol is contained in an amount of 12 wt % or higher, preferably 18 wt % or higher in the polyurethane prepolymer composition. Since such polyurethane prepolymer has a high hydrophilicity, the reaction completes in an early time while generating the heat reaction. Further, for the increase of the initial curability, the catalyst amount is influential in addition to the water permeability. Accordingly, as the catalyst amount, it is preferably added to the polyurethane prepolymer composition so that the gelling time is 70 seconds or lower.

### EXAMPLES

The embodiments of the present invention will be explained further specifically with reference to examples and comparative examples. However, it should be mentioned that the present invention is by no means restricted thereto and those skilled in the art can understand various other embodiments.

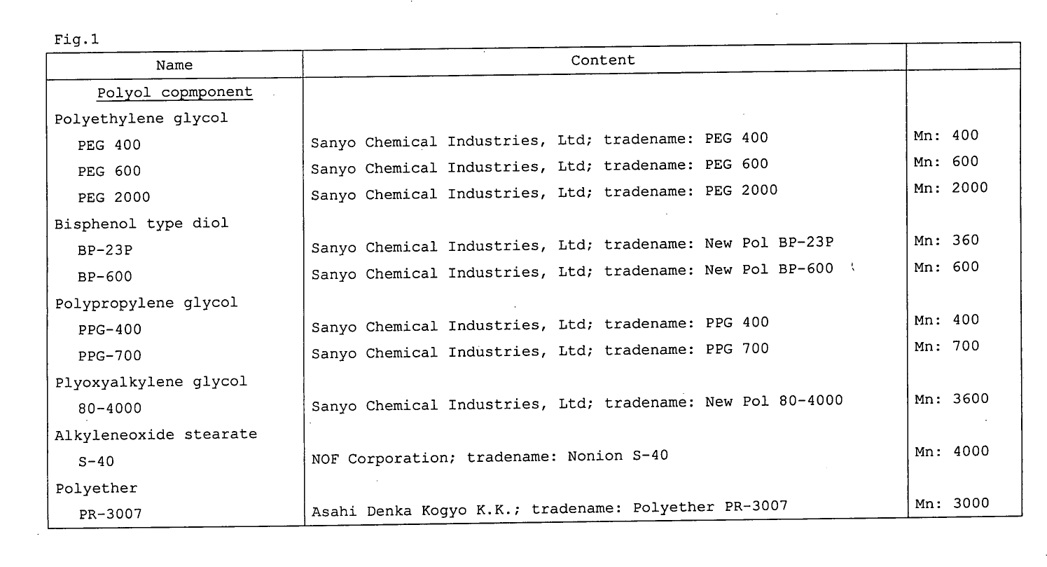

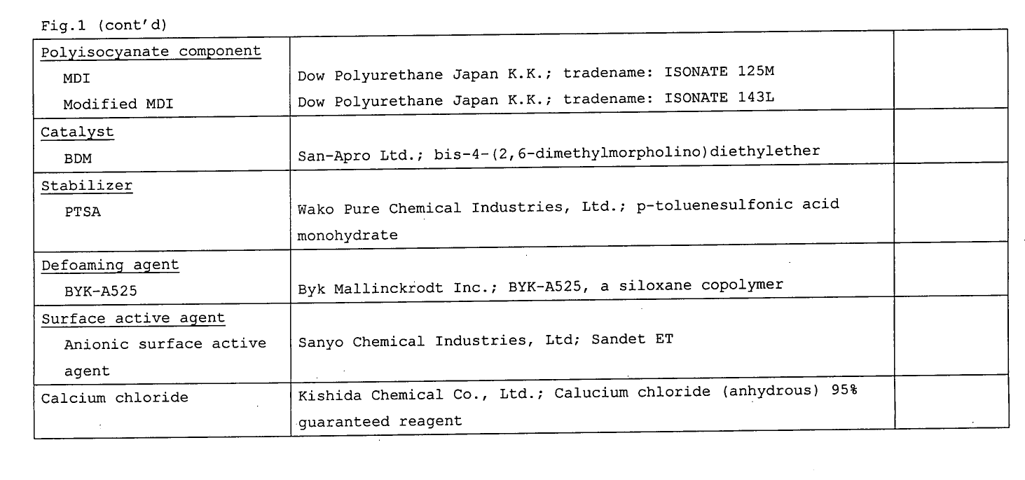

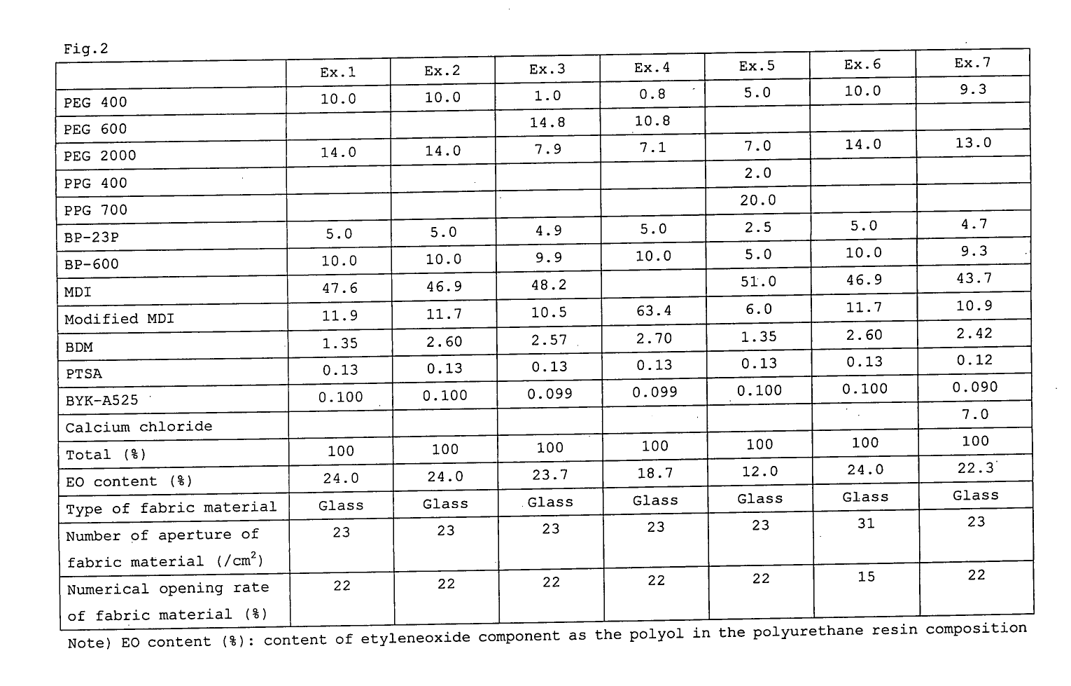

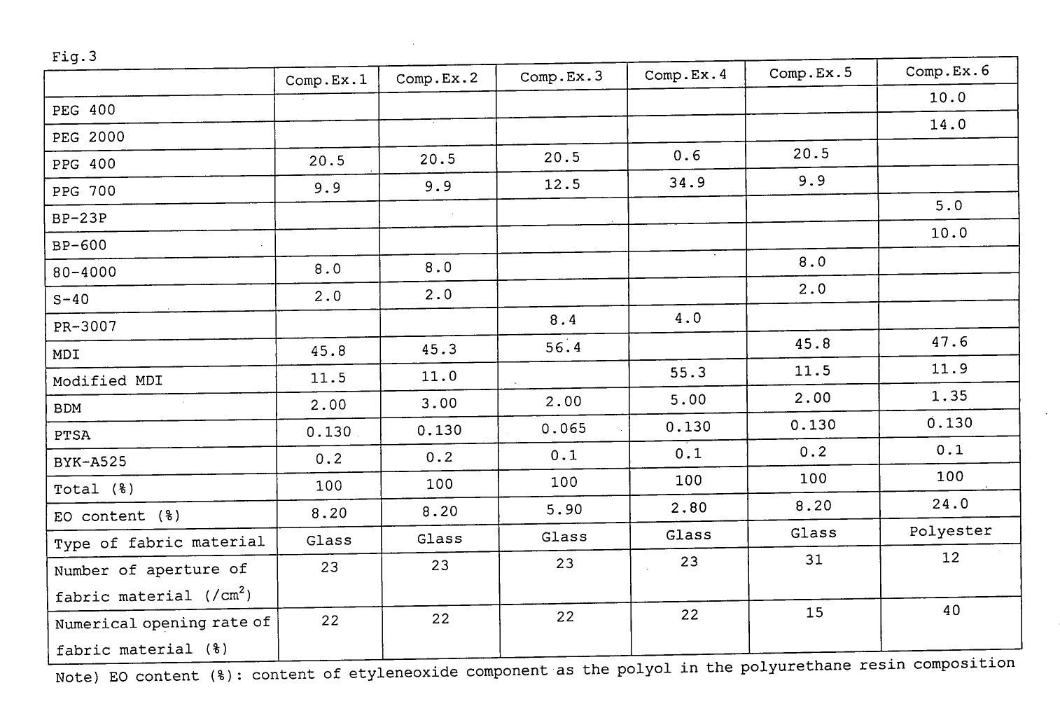

The materials used for the polyurethane prepolymer compositions of the examples and comparative examples are indicated in FIG. 1, and the formulations are indicated in FIG. 2 and FIG. 3. Further, the physical properties of the polyurethane prepolymer compositions obtained from these formulations and the water-curable supporting bandages coated on the fabric material were evaluated with respect to the following items.

In preparation of the polyurethane prepolymer compositions, in all of the examples and comparative examples, a polyol component and a defoaming agent were charged into a reaction container flushed with nitrogen gas, and the water was removed at from 80 to 100° C., and then a part of a stabilizer was added. Then, a polyisocyanate component was added thereto, and stirring was conducted at from 70 to 80° C. for about 3 hours. Further, a catalyst and the rest of the stabilizer were added, and stirring was conducted for one hour to obtain a polyurethane prepolymer composition. This polyurethane prepolymer composition was sealed in a container which was flushed with nitrogen gas.

In all of the examples and comparative examples, preparation of the water-curable supporting bandage was conducted by coating the composition on a fiber material. As the fiber material, a tape-shaped fiber material obtained by raschel fabrication of glass fibers to have a number of apertures (mesh size) of 23 per cm², a numerical opening rate of aperture of 22% and a width of 100 mm, and further heat-cleaning, was used. Further, one which was the same as above provided that the number of apertures of 31 per cm² and the numerical opening rate of aperture of 15%, was used. Further, in one of the comparative example, a tape-shaped fabric material obtained by raschel fabrication of polyester fibers under the conditions that a number of apertures of 12 per cm² and a numerical opening rate of aperture of 40%, and heat-setting, was used.

The coating of the polyurethane prepolymer composition on this fabric material was carried out by a roll coater method. The polyurethane prepolymer composition was coated on the glass fabric material in 220 g/m² on average. Further, it was coated on the polyester fabric material in 280 g/m² on average. The fabric material on which the polyurethane prepolymer composition was coated was wound up in a length of 3.6 m to obtain a water-curable supporting bandage, and sealed in a moisture-impermeable bag which was flushed with nitrogen gas.

Evaluation of the physical properties of the polyurethane prepolymer compositions and the water-curable supporting bandage were conducted as follows,respectively. In a spray method, unless otherwise specifically described, a 1 wt % aqueous solution having an anionic surface active agent, i.e. polyoxyethylene laurylether sulfate ethanolamine (manufactured by Sanyo Chemical Industries, Ltd; “Sandet ET”) added to water, was used. As the spray method, spraying was made by a sprayer so that the composition would be attached uniformly in an amount of 40 g to 1 m² of the water-curable supporting bandage.

The evaluation of the water-curable supporting bandage was made with respect to the following items.

Working Time in an Immersion Method

In a measurement room controlled to have a room temperature of 23° C. and a humidity of 65% RH, the water-curable supporting bandage was taken out of the moisture-impermeable bag, and immersed in water of 20° C. for 10 seconds, and lightly drained. This water-curable supporting bandage was wound around the outer periphery of a cylinder, and the time when rolling can no longer be made was measured.

Working Time in a Spray Method

In a measurement room controlled to have a room temperature of 23° C. and a humidity of 65% RH, the water-curable supporting bandage was taken out of the moiture-impermeable bag, and wound as it is around the outer periphery of a cylinder, and the time when rolling can no longer be made was measured.

Heat Generation Temperature in a Spray Method

In a measurement room controlled to have a room temperature of 20° C. and a humidity of 20% RH, a temperature sensor was provided on the surface of a polyethylene container of 85 mm in diameter which was preliminarily warmed to 37° C. by hot water. On this polyethylene container, a cast padding of nonwoven fabric having a thickness of 3 mm was wound in two layers, and further thereon, a water-curable supporting bandage taken out of the moisture-impermeable bag was wound in 13 layers. Over the 13 layers round up water-curable supporting bandage, water containing a surface active agent was sprayed on the surface of wound bandage by a sprayer evenly throughout it, and the maximum heat-geneation temperature was measured.

Change of Strength with the Lapse of Time in an Immersion Method (Initial Curability in an Immersion Method)

In a measurement room controlled to have a room temperature of 20° C. and a humidity of 20% RH, the water-curable supporting bandage was taken out of the moisture-impermeable bag,and dipped in water of 20° C. for 5 seconds. At this time, the bandage was taken out without squeezing it, and lightly taken, and swung 3 times to drain the water. Then, the water-curable supporting bandage was wound in 5 layers without applying tension to the bandage around an acryl cylindrical container on which a cast padding was preliminarily wound and which was 60 m in diameter, controlled at 32° C. and filled with warmed water. And, while taking a care not to let it shift in a transverse direction, the surface was rubbed to form a cylindrical sample. After 5 minutes had passed,the sample was drawn out from the acryl cylindrical container without causing deformation, and the sample was mounted on a jig having distance of 50 mm between supporting points. This sample was compressed in a radial direction by Autograph AG-D (manufactured by Shimadzu Corporation; a precision universal testing machine of a computer-controlled measurement type), and the compressive strength until deformation of 5 mm was observed was measured. Likewise, after 7 minutes had passed and 10 minutes had passed, other samples prepared in the same way were measured. The test speed was 25 mm/min.

Change of Strength with the Lapse of Time in a Spray Method (Initial Curability in a Spray Method)

In a measurement room controlled to have a room temperature of 20° C. and a humidity of 20% RH, the water-curable supporting bandage taken out of the bag was wound in 5 layers without applying tension to the bandage around an acryl cylindrical container on which a cast padding was preliminarily wound and which was 60 mm in diameter, controlled at 32° C. and filled with warmed water. Over the surface of the roll, the water containing a surface active agent was sprayed by a sprayer evenly, and while taking a care not to let it shift in a transverse direction, the surface was rubbed to form a cylindrical sample. After 5 minutes had passed,the sample was drawn out from the acryl cylindrical container without causing deformation, and the compressive strength was likewise measured by Autograph AG-D. Likewise, after 7 minutes had passed and 10 minutes had passed, other samples prepared in the same way were measured.

Compressive Strength One Day After

In the same manner as the above initial curability, samples were prepared for the immersion method and the spray method, and 15 minutes after, these were drawn out without causing deformation. After these were left to stand in a constant temperature incubator of 20° C. for one day, the compressive stregth was likewise measured by Autograph AG-D. The test speed was 25 mm/min.

Gelling Time

In a measurement room controlled to have a room temperature of 20° C. and a humidity of 20% RH, 5 g of a polyurethane prepolymer composition was weighed and poured into a vessel (beaker), and 1 g of water was added thereto, followed by stirring with a glass rod for 20 seconds. Then, the resin surface elevated in the vessel with bubbling of preplolymer composition, and the time from the start of stirring until the bubble is broken at around the portion where the elevation stopped was measured and it was called a gelling time (seconds).

Results of Measurement

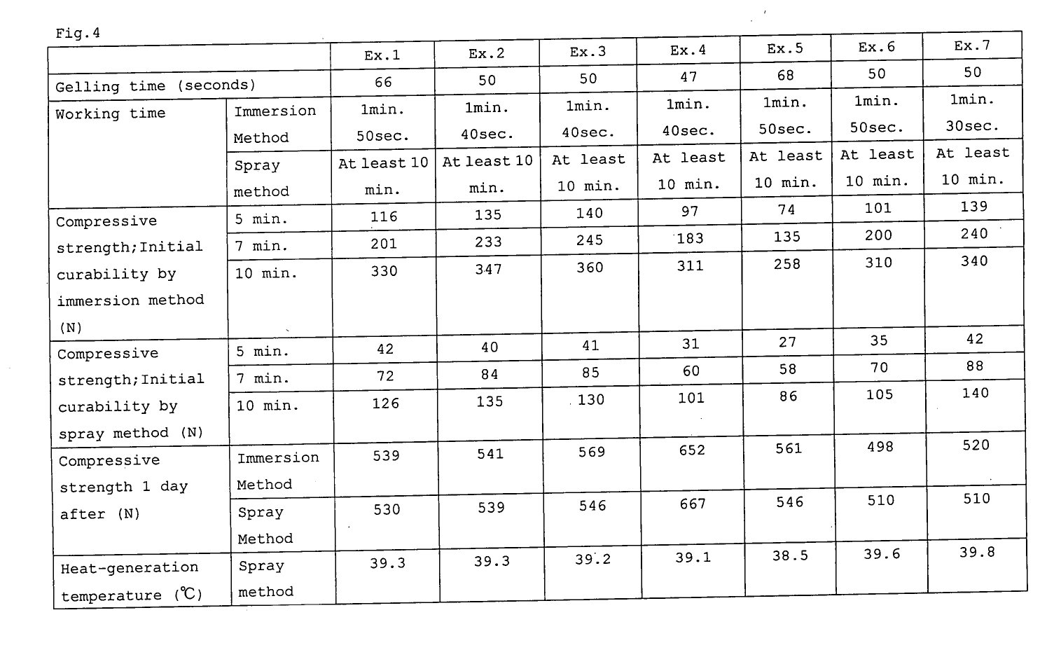

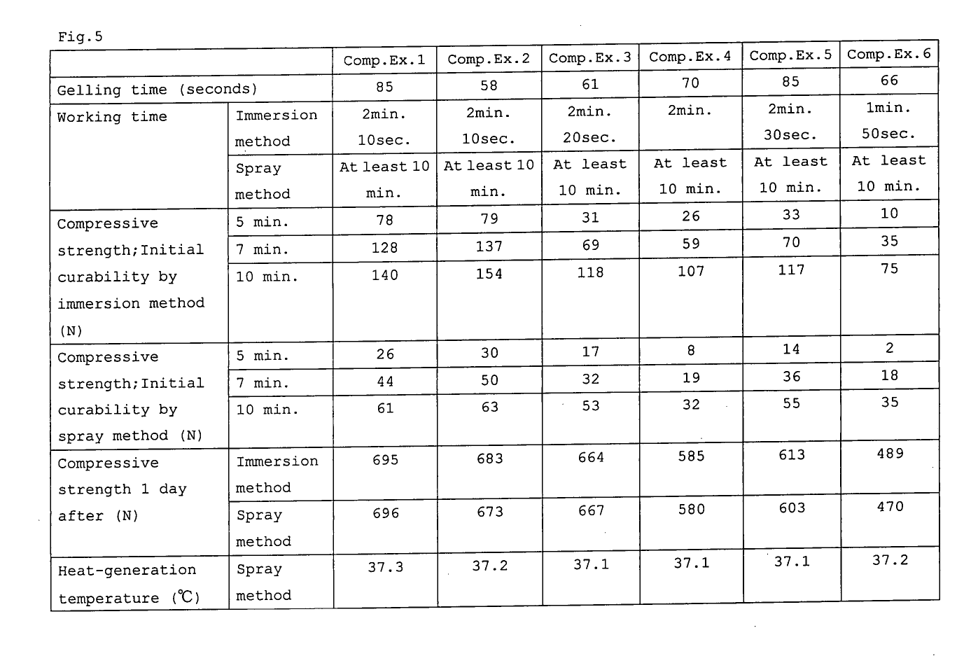

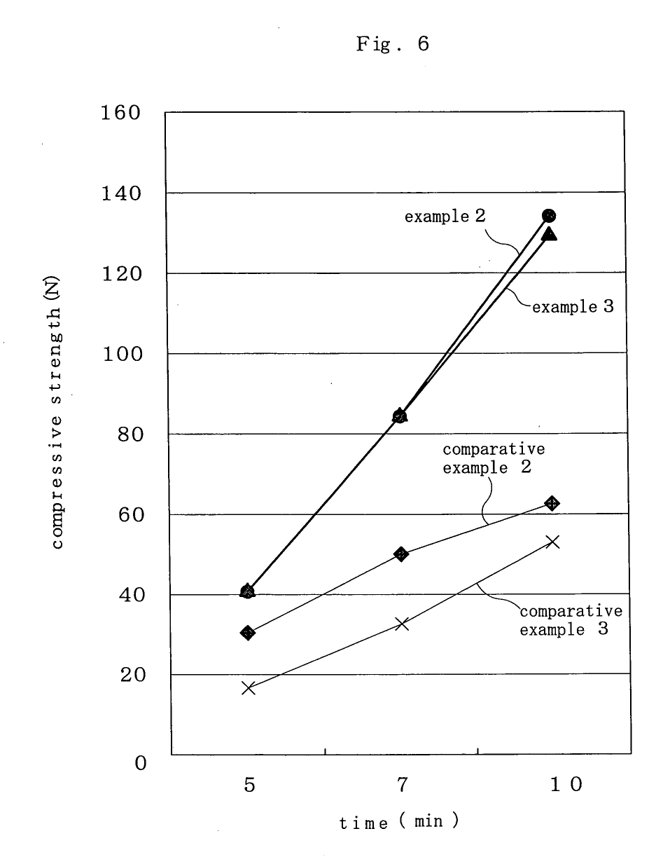

The results of respective measurements concerning the physical properties and evaluation in examples and comparative examples are indicated in FIG. 4 and FIG. 5. Further, the relations between the elapsed time and the strength concerning the initial curability for respective spray methods in Examples 2 and 3 and Comparative Examples 2 and 3 are indicated in FIG. 6. With respect to the above measurement results, explanation will be given below.

Working Time

In respective Examples of the present invention, since the working time by the spray method is 10 minutes or longer, it is possible to securely apply the water-curable supporting bandage to complicated sites of the affected part with sufficient time. On the other hand, in the immersion method of Comparative Examples, since the working time is from 1 minute 50 seconds to 2 minutes 30 seconds, this time is too short to apply the water-curable supporting bandage to complicated sites and the supporting bandage may sometimes be cured before the completion of application. Here, if the ones in Examples are applied to the immersion method, since the working time is as short as from 1 minute 30 seconds to 1 minute 50 seconds, it is difficult in the immersion method to take sufficient time for the application to the affected part.

Initial Curability

It is commonly known that the initial curability is not at a practical level if the strength does not reach about 78 N or higher at the point of 10 minutes later. In the ones made by the spray method of all of Examples, the strength is within a range of from 86 to 140 N i.e., over 78 N, and it is confirmed that these have an appropriate initial curability.

On the other hand, the ones in Comparative Examples 1 to 5, the strength is from 107 to 154 N by the immersion method,such being appropriate. However,by the spray method, the strength is from 32 to 63 N i.e. does not reach 78 N, and it is therefore concluded that the use by the spray method is inconvenient. Further, by the one in Comparative Example 6, although sufficient strength can be obtained in the after-mentioned compressive strength one day after,no sufficient initial curability can be obtained in both the immersion method and the spray method. Furthermore, in the ones in all of Examples, in the immersion method, the strength exceeds 78 N in an early time at a level of about 5 minutes, and the curing is too fast.

Compressive Strength One Day After

In Examples and Comparative Examples, sufficient compressive strength can be seen in both the spray method and the immersion method, and therefore no difference is seen in this point.

Heat-generation Temperature

In the spray method, the maximum temperature in Examples is from 38.5 to 39.8° C., and this heat generation is within an allowable range and no inconvenience is caused. On the other hand, in Comparative Examples, the heat-generation temperature is low, and the reaction does not proceed at an appropriate speed, and the the low numerical value of the initial curability reflects this result.

Gelling Time

In respective Examples and Comparative Examples, the gelling times in Comparative Examples 1 and 5 are over 70 seconds and considered to be too long, but other ones including respective Examples are within an appropriate range.

Total Evaluation

In the ones of Examples, in the case of the spray method, sufficient working time is obtained, appropriate numerical values are obtained in the initial curability and the compressive strength one day after, and the heat-generation temperature is appropriate, whereby these can be used adequately.

In the ones of Comparative Examples, in the case of the spray method, the working time is long, but the heat-generation temperature is low, whereby no sufficient initial curability can be obtained. Further, in the case of the immersion method, it is considered that the initial curability and the compressive strength one day after are within appropriate ranges, but the working time is too short, such being problematic in use.

### INDUSTRIAL APPLLICABILITY

In the present invention, in the case where the water is supplied by a spray method for curing, the working time is 10 minutes or longer as mentioned above. Accordingly, even when it is applied the an affected part of complicated shape of a patient, curing is not advanced in the halfway of application, and sufficient time can be used. Further, since the water for curing is supplied from the outside after the water-curable supporting bandage is wound in several layers, the water amount which reaches the affected part is small, and the inner side is not substantially wetted, whereby the heat generation at the affected part side is not so high. Accordingly, unpleasantness of patients can be reduced.

Further, since this bandage ean be cured with a small amount of water by the spray method, it is not necessary to prepare a large amount of water in a bucket like the immersion method. And, if an aerosol container or the like filled with water is prepared, this bandage can be used anywhere wihtout dirtying floor. Accordingly,it can be effectively used for treatment of e.g., bone fracture in a site where water can not be prepared easily such as disaster-stricken district.

1. A water-curable suppoprting bandage, wherein:
the water-curable suppoprting bandage is capable of curing by spraying a small volume of water of from 20 to 60 g/m² from outside after winding the bandage around an injured or diseased part (an affected part) of patient;
the supporting bandage is formed by a relatively coarse and flexible fabric material coated by the water-curable polyurethane prepolymer composition which is cured in a short time;
the water-curable polyurethane prepolymer composition contains a polyurethane prepolymer containing a polyol component and polyisocyanate, and a catalyst;
an ethyleneoxid component of the polyol component is contained in an amount of 12 wt % or higher in the water-curable polyurethane prepolymer composition; and
the fabric material of the supporting bandage has from 20 to 25 apertures per cm² and a numerical opening rate of aperture of from 18 to 27% so as to increase a permiability of the water to be sprayed.

2. The water-curable supporting bandage according to claim 1, wherein the water to be sprayed from the outside after application to the affected part contains a surface active agent so as to uniformly impregnate the water to the water-curable polyurethane prepolymer composition and accelerate the curing thereof.

3. The water-curable supporting bandage according to claim 2, wherein the surface active agent is a nonionic or anionic surface active agent and has a low skin stimulus property.

4. (deleted)

5. (deleted)

6. The water-curable supporting bandade according to any one of claims 1 to 3, wherein the water-curable polyurethane prepolymer composition contains at least one of a chloride of a metal, an oxide of a metal, a sulfate of a metal, and silica which are capable of generating heat when it contacts water, to accelerate the curing of the polyurethane prepolymer.

7. (deleted)

8. The water-curable supporting bandage according to claim 6, wherein the chloride of a metal is any one of calcium chloride and magnesium chloride.

9. The water-curable supporting bandage according to claim 6, wherein the oxide of a metal is any one of calcium oxide and zinc oxide.

10. The water-curable supporting bandage according to claim 6, wherein the sulfate of a metal is any one of calcium sulfate and magnesium sulfate.

11. The method for using a water-curable supporting bandage, comprising:
preparing a water-curable supporting bandage which is formed by a relatively coarse and flexible fabric material coated with a water-curable polyurethane prepolymer composition which is capable of curing in a short time;
the water-curable polyurethane prepolymer composition contains a polyurethane prepolymer containing a polyol component and polyisocyanate, and a catalyst;
an ethyleneoxid component of the polyol component is contained in an amount of 12 wt % or higher in the water-curable polyurethane prepolymer composition;
the fabric material of the supporting bandage has from 20 to 25 apertures per cm² and a numerical opening rate of aperture of from 18 to 27%;
pre-winding a cast padding around the injured or diseased part;
winding the water-curable supporting bandage over the cast padding;
spraying a small amount of water of from 20 to 60 g/m² from outside of the water-curable supporting bandage; and
curing the water-curable supporting bandage.

12. The method for using a water-curable supporting bandage according to claim 11, wherein the water to be sprayed from the outside contains a surface active agent as to uniformly impregnate the water in the water-curable polyurethane prepolymer composition and accelerate the curing thereof.

13. The method for using a water-curable supporting bandage according to claim 12, wherein the surface active agent is a nonionic or anionic surface active agent and has low skin stimulus property.

14. (deleted)

15. (deleted)
